(19) United States
(12) Patent Application Publication
(10) Pub. No.: US 2012/0270904 A1
(43) Pub. Date: Oct. 25, 2012

(54) CO-CRYSTAL OF 4-FURAN-2(5H)-ONE WITH OXALIC ACID AND USE THEREOF AS PESTICIDE

(75) Inventors: Martin Weiss, Bornheim,/Sechtem (DE); Dirk Storch, Koln (DE); Wolfgang Wirth, Bergisch Gladbach (DE); Britta Olenik, Bottrop (DE); Ulrich Schwiedop, Monheim (DE); Hans-Christoph Weiss, Essen (DE)

(73) Assignee: BAYER TECHNOLOGY SERVICES GMBH, Leverkusen (DE)

(21) Appl. No.: 13/504,029

(22) PCT Filed: Oct. 26, 2010
(86) PCT No.: PCT/EP2010/066094

§ 371 Date: Jul. 11, 2012

(30) Foreign Application Priority Data
Oct. 27, 2009 (EP) 09013508.8

(51) Int. Cl. C07D 405/12; A01P 5/00; A01P 7/04; A01P 7/00; A01N 43/40; A01P 15/00

(52) U.S. Cl. 514/336; 546/284.4

(57) ABSTRACT

The invention relates to a new co-crystal of 4-{[(6-chloropyrid-3-yl)methyl](2,2-difluoroethyl)amino}furan-2(5H)-one with oxalic acid, and also to processes for preparation thereof and use.

The invention relates to a novel cocrystal of 4-{[(6-chloropyrid-3-yl)methyl](2,2-difluoroethyl)amino}furan-2(5H)-one with oxalic acid, and to processes for production thereof and use thereof.

The compound 4-{[(6-chloropyridin-3-yl)methyl](2,2-difluoroethyl)amino}furan-2(5H)-one and methods for preparation of this compound are known. It is also known that this compound has insecticidal and/or acaricidal action. For example, WO 2007/115644 A1 gives a first description of the preparation of this compound and use thereof for control of arthropods, especially insects. The preparation of this compound has also been described in WO 2009/036899 A1. It has now been found that the compounds prepared by the known process cannot be employed in an economically relevant form.

It is known that solids may be present in amorphous and crystalline form and as polymorphs, hydrates and solvates, which can have a significant influence especially on physical properties, such as solubility, bioavailability on uptake into an organism, hygroscopicity and melting point. These physical properties can limit or extend the usability of the substance in one way or another.

It is additionally known that, for some polymorphs, a particular modification constitutes the thermodynamically stable phase over the entire temperature range up to the melting point, whereas, for other systems, there exist one or more transition points at which the stability relationship is reversed. It is not possible to predict the stability relationship and, more particularly, the existence and position of transition points referred to above. There is a review of the state of knowledge about these fundamental thermodynamic relationships in J. Bernstein, R. J. Davey, J. O. Henck, Angew. Chem. Int. Ed., 1999, 38, 3440-3461.

The occurrence of active ingredients in various crystalline modifications is of great significance both for the development of production processes and for the development of formulations. For instance, the different crystalline modifications of a chemical compound differ not only in appearance (crystal habit) and hardness, but also in numerous further physicochemical properties. Differences in terms of stability, filterability, grindability, solubility, hygroscopicity, melting point, solid density and flowability can exert a strong influence on the quality and the efficacy of plant treatment compositions. It has not been possible to date to predict the occurrence and number of crystalline modifications, including the physicochemical properties thereof. In particular, the thermodynamic stability and also the different behavior after administration in living organisms cannot be determined in advance.

A further crystalline modification of a solid is that of what are called cocrystals. These comprise the solid with what are called coformers. Here too, advantageous physical properties compared to the original solid can be obtained.

For example, WO 2008/013823 A2 describes a process for producing a cocrystal comprising (2R-trans)-6-chloro-5[[4-[(4-fluorophenyl)methyl]-2,5-dimethyl-1-piperazinyl]carbonyl]-N,N, 1-trimethyl-alpha-oxo-1H-indol-3-acetamide or the hydrochloride thereof or the free base thereof and a coformer, which may be arginine, urea, salicylic acid, 4-aminosalicylic acid and benzoic acid. The cocrystals disclosed may also contain more than one coformer. The stoichiometric ratio in which crystalline solid and coformer may be present relative to one another may be 1:1, 2:1 or 1:2. It is additionally stated that the cocrystals found have an advantageous solubility, dissolution rate, bioavailability, stability and further processability.

The process disclosed in WO 2008/013823 A2 for producing the cocrystals may be characterized by grinding of the crystalline solid together with the at least one coformer or combined melting of the crystalline solid with the at least one coformer, or alternatively by combined dissolution of crystalline solid with the at least one coformer and subsequent crystallization. In this context, the crystalline substance and the at least one coformer may be in a stoichiometric ratio of 1:1 to 1:100 relative to one another. The aforementioned grinding of the crystalline solid together with the at least one coformer can also be performed together with a small amount of solvent.

The processes and apparatuses disclosed in WO 2008/013823 A2 for characterization of the cocrystal formed include thermogravimetric analysis (TGA), powder x-ray diffractometry analyses (p-XRD) and differential calorimetry (DSC).

WO 2008/013823 A2 does not disclose a cocrystal comprising 4-{[(6-chloropyrid-3-yl)methyl](2,2-difluoroethyl)amino}furan-2(5H)-one. WO 2008/013823 A2 further discloses that it is generally possible for cocrystals of a crystalline solid to feature an improvement in the aforementioned physical properties, but this is not explained in connection with the cocrystals disclosed in WO 2008/013823 A2. It additionally appears to be doubtful whether the cocrystals disclosed actually exist. What are disclosed are merely indications that the cocrystals could exist. No ultimate proof by the growing of a single crystal and analysis thereof is disclosed. The powder x-ray diffractometry analyses disclosed can also transport incorrect information as a result of diffraction and refraction at the particle surfaces.

US 2007/0212683 A1 discloses a cocrystal of VX-950, a hepatitis C virus inhibitor, with oxalic acid among other substances.

Similarly to the disclosure of WO 2008/013823 A2, it is disclosed in general terms here too that the resulting cocrystals can have advantageous physical properties. A stability measurement a suspension of the cocrystal is disclosed, but no comparative disclosure between the pure crystalline solid and the cocrystals can be found. Furthermore, in the disclosure of US 2007/0212683 A1 too, the existence of the cocrystals disclosed may be doubtful, since only powder x-ray diffractometry analyses were conducted here too, with the error sources which result therefrom.

WO 2008/096005 A1 describes thiophanate-methyl as a coformer for agrochemically active substances which must include at least one functional group which acts as a hydrogen acceptor of a hydrogen bond.

In contrast to WO 2008/013823 A2 and US 2007/0212683 A1, single crystals of some cocrystals are detected in WO 2008/096005 A1. A single crystal of a cocrystal comprising 4-{[(6-chloropyrid-3-yl)methyl](2,2-difluoroethyl)amino}furan-2(5H)-one is, however, not described. It is disclosed at the same time that the formation of such cocrystals can neither be tailored nor predicted. It can therefore be assumed that the disclosure of WO 2008/096005 A1, like that of WO 2008/013823 A2 and US 2007/0212683 A1 and further prior art too, which does not disclose the presence of a cocrystal of 4-{[(6-chloropyrid-3-yl)methyl](2,2-difluoroethyl)amino}furan-2(5H)-one by means of the detection of a single crystal, cannot anticipate or suggest a cocrystal of 4-{[(6-chloropyrid-3-yl)methyl](2,2-difluoroethyl)amino}furan-2(5H)-one.

Proceeding from this prior art and the shortcomings found therein, it is thus an object of the present invention to provide a cocrystal which alters the physical properties of 4-{[(6-chloropyrid-3-yl)methyl](2,2-difluoroethyl)amino}furan-2(5H)-one such that the above advantages are obtained.

As a first part of the subject matter of this invention, it has now been found that, surprisingly, this object is achieved by a cocrystal comprising 4-{[(6-chloropyrid-3-yl)methyl](2,2-difluoroethyl)amino}furan-2(5H)-one, which is characterized in that it comprises oxalic acid as a coformer.

In connection with the present invention, cocrystal refers to a substance which is solid at room temperature (23° C.) and ambient pressure (1013 hPa), and which comprises, in its crystal lattice, at least two pure substances which interact with one another through hydrogen bonding, all pure substances present in the crystal lattice likewise being solids at room temperature (23° C.) and ambient pressure (1013 hPa).

The term “coformer” as used in connection with the present invention refers to a pure substance which is not 4-{[(6-chloropyrid-3-yl)methyl](2,2-difluoroethyl)amino}furan-2(5H)-one and which, together with 4-{[(6-chloropyrid-3-yl)methyl](2,2-difluoroethyl)amino}furan-2(5H)-one in at least one stoichiometric ratio, forms an adduct having only one melting point.

4-{[(6-Chloropyrid-3-yl)methyl](2,2-difluoroethyl)amino}furan-2(5H)-one refers, in connection with the present invention, to all polymorphs, solvates and also hydrates of the substance of the formula (I):

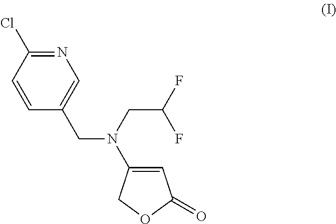

The inventive cocrystal typically has an increase in melting point compared to the pure substance 4-{[(6-chloropyrid-3-yl)methyl](2,2-difluoroethyl)amino}furan-2(5H)-one.

“Melting point” in the context of the present invention is understood to mean that temperature at which the substance has the highest heat release when the melting point is measured in differential calorimetry (DSC). The melting of the substance already sets in at an earlier stage than that temperature at which the aforementioned highest heat release can be measured, and typically also ends at higher temperatures, which is why the melting points reported hereinafter may quite possibly also be at lower or higher temperatures according to other definitions of the melting point.

The elevated melting point of the inventive cocrystal is particularly advantageous because the cocrystal, in contrast with the pure substance 4-{[(6-chloropyrid-3-yl)methyl](2,2-difluoroethyl)amino}furan-2(5H)-one, can be treated at higher temperatures without melting.

Such a treatment at higher temperatures may be drying in the course of production of the cocrystal, or else storage at elevated temperatures.

More particularly, the inventive cocrystal exhibits improved storage stability since, in the course of storage at elevated temperatures, it does not tend to partially melt at its surface and hence to agglomerate, or adhere to the wall of the transport vessel in which it is transported.

In addition, the inventive cocrystal typically has an elevated solubility in water compared to the pure substance 4-{[(6-chloropyrid-3-yl)methyl](2,2-difluoroethyl)amino}furan-2(5H)-one.

An elevated solubility in water is advantageous especially when the 4-{[(6-chloropyrid-3-yl)methyl](2,2-difluoroethyl)amino}furan-2(5H)-one is to be sprayed in the use thereof. It is thus possible to produce more highly concentrated solutions of 4-{[(6-chloropyrid-3-yl)methyl](2,2-difluoroethyl)amino}furan-2(5H)-one, which are correspondingly more effective.

In a first preferred development of the inventive cocrystal, the cocrystal consists essentially of 4-{[(6-chloropyrid-3-yl)methyl](2,2-difluoroethyl)amino}furan-2(5H)-one and oxalic acid as a coformer.

The melting point of the inventive cocrystal is typically in the range from 110° C. to 130° C., preference being given to inventive cocrystals having a melting point of about 122° C.

The aforementioned melting point can be determined in a commonly known manner by differential calorimetry (DSC).

Typically, the stoichiometric ratio of 4-{[(6-chloropyrid-3-yl)methyl](2,2-difluoroethyl)amino}furan-2(5H)-one to oxalic acid in the first preferred development is 2:1.

The inventive cocrystal according to the first preferred development is further characterized in that it has monoclinic morphology. At the same time, the cocrystal typically has the space group C2/c according to the Cambridge Structural Database (F. H., Allen, Acta Cryst.B58, (2002) 380-388).

The aforementioned morphology and space group, and also the aforementioned stoichiometric ratio of 4-{[(6-chloropyrid-3-yl)methyl](2,2-difluoroethyl)amino}furan-2(5H)-one to oxalic acid, can be determined in a commonly known manner by x-ray diffraction analysis of a single crystal of the cocrystal.

The present invention further provides a first process for producing a cocrystal comprising 4-{[(6-chloropyrid-3-yl)methyl](2,2-difluoroethyl)amino}furan-2(5H)-one, characterized in that it comprises at least the steps of
a) admixing 4-{[(6-chloropyrid-3-yl)methyl](2,2-difluoroethyl)amino}furan-2(5H)-one with oxalic acid to obtain a mixture A,
b) optionally further admixing the mixture A with a solvent to obtain a mixture A′ and
c) grinding mixture A or mixture A′ to obtain the inventive cocrystal.

The admixing according to step a) of the process according to the invention can be accomplished either by initially charging 4-{[(6-chloropyrid-3-yl)methyl](2,2-difluoroethyl)amino}furan-2(5H)-one and adding oxalic acid, or vice versa. Preference is given to initially charging 4-{[(6-chloropyrid-3-yl)methyl](2,2-difluoroethyl)amino}furan-2(5H)-one and metering in oxalic acid while weighing.

The mixture A obtained in step a) of the process according to the invention may comprise 4-{[(6-chloropyrid-3-yl)methyl](2,2-difluoroethyl)amino}furan-2(5H)-one and oxalic acid in any desired stoichiometric ratios.

For the production of the first preferred development of the inventive cocrystal, the mixture A preferably comprises 4-{[(6-chloropyrid-3-yl)methyl](2,2-difluoroethyl)amino}furan-2(5H)-one in a stoichiometric ratio of 2:1 relative to oxalic acid.

This also gives rise to the preferred procedure of metered addition of oxalic acid to 4-{[(6-chloropyrid-3-yl)methyl](2,2-difluoroethyl)amino}furan-2(5H)-one while weighing. With this procedure, it is possible in a particularly exact manner to establish the aforementioned stoichiometric ratio relative to 4-{[(6-chloropyrid-3-yl)methyl](2,2-difluoroethyl)amino}furan-2(5H)-one, such that the cocrystal according to the first preferred development forms preferentially with its particularly advantageous properties.

In a preferred embodiment of the process, the admixing is effected according to step b).

The solvent used in step b) of the process according to the invention may be any suitable solvent which is capable of dissolving at least one of the substances oxalic acid and/or 4-{[(6-chloropyrid-3-yl)methyl](2,2-difluoroethyl)amino}furan-2(5H)-one in a concentration of at least 0.01 g/l. Suitable solvents are especially acetone, ethyl acetate, ethanol and 2-propanol, and mixtures thereof.

In step b) of the process according to the invention, the solvent is added in such an amount that the solubility concentration (solubility) of 4-{[(6-chloropyrid-3-yl)methyl] (2,2-difluoroethyl)amino}furan-2(5H)-one and/or oxalic acid in the resulting mixture A′ is exceeded. Preferably, the solubility concentration of 4-{[(6-chloropyrid-3-yl)methyl] (2,2-difluoroethyl)amino}furan-2(5H)-one and oxalic acid is exceeded. This is particularly advantageous because, as a result, the solids are essentially still present as solids and only the surfaces of the solids are wetted with the solvent. This improves the molecular contact between the 4-{[(6-chloropyrid-3-yl)methyl](2,2-difluoroethyl)amino}furan-2(5H)-one and the oxalic acid coformer, without disproportionately increasing the probability of formation of solvates and/or hydrates. Moreover, as a result, the presence of the solvent cannot significantly counteract molecular adduct formation in the manner of hydrogen bonding between 4-{[(6-chloropyrid-3-yl)methyl](2,2-difluoroethyl)amino}furan-2(5H)-one and the respective coformer.

The grinding of mixture A or mixture A′ can be effected in all apparatuses which are commonly known to the person skilled in the art and are suitable for grinding of solids.

Nonexclusive examples of apparatuses in which such grinding can be executed include, for instance, mortar mills, vibratory mills or ball mills. Preference is given to executing step c) of the process according to the invention in vibratory mills or ball mills.

In the case of such grinding processes, the energy introduced into the material to be ground must be such that there is no unwanted formation of amorphous phases of the solids, but there is at the same time intensive contacting of the solids in the grinding apparatus. There are thus upper and lower limits to the range of those energies which can be introduced into the solids by means of the grinding apparatus.

The person skilled in the art is aware of suitable parameters with which he can establish this energy. These include, for instance, the duration and intensity (for example contact pressure of a mortar mill or size and material, and amplitude and frequency in a vibratory mill or rotational speed in a ball mill) of grinding.

Such procedures are particularly advantageous because the limitation of the energy introduced can ensure, with regard to the minimum level of energy, a minimum level of intensive contacting of 4-{[(6-chloropyrid-3-yl)methyl](2,2-difluoroethyl)amino}furan-2(5H)-one with the oxalic acid coformer, while the upper limit simultaneously prevents excessive stress on the substances. The result of such excessive stress on the substances which are ground may be that they are converted to an amorphous state, which can possibly prevent the formation of a cocrystal, or destroy any cocrystal formed.

The entire process is typically executed at room temperature (23°) and ambient pressure (1013 hPa). It may be appropriate, however, to execute the process and especially step c) at lower temperatures, in order to be able to tolerate a certain degree of heating of the substances to be ground, without the aforementioned conversion to the amorphous state as a result of the energy input.

The present invention further provides a second process for producing a cocrystal comprising 4-{[(6-chloropyrid-3-yl)methyl](2,2-difluoroethyl)amino}furan-2(5H)-one, characterized in that it comprises at least the steps of
a) providing a saturated solution (A) of 4-{[(6-chloropyrid-3-yl)methyl] (2,2-difluoroethyl)amino}furan-2(5H)-one and/or a saturated solution (B) of oxalic acid, and
b) admixing solutions (A) and (B) and/or or adding oxalic acid as a solid to solution (A) and/or adding 4-{[(6-chloropyrid-3-yl)methyl](2,2-difluoroethyl)amino}furan-2(5H)-one as a solid to solution (B).

In the second process according to the invention above, solution (A) can be produced with a first solvent and solution (B) with a second solvent.

The first and second solvents may independently be selected from a list consisting of acetone, ethyl acetate, ethanol, 2-propanol and water, or mixtures thereof.

In preferred embodiments, the first and second solvents are the same.

In step a) of the second process according to the invention, it is also possible for only a solution (A) or a solution (B) to be present. When only a solution (A) or a solution (B) according to step a) of the process is provided, in step b), either oxalic acid in solid form is added to solution (A) or 4-{[(6-chloropyrid-3-yl)methyl](2,2-difluoroethyl)amino}furan-2(5H)-one in solid form is added to solution (B).

It is also possible in alternative embodiments of the second process according to the invention to provide a solution (A) and a solution (B) according to step a), and to admix them according to step b) of the process, while oxalic acid in solid form and/or 4-{[(6-chloropyrid-3-yl)methyl](2,2-difluoroethyl)amino}furan-2(5H)-one in solid form is also added to solutions (A) and (B) being admixed.

Preference is given to executing the second process according to the invention in such a way that, in step a), solutions (A) and (B) are provided and these solutions are admixed with one another, without addition of further solid.

The present invention further provides for the use of the inventive cocrystals of 4-{[(6-chloropyrid-3-yl)methyl](2,2-difluoroethyl)amino}furan-2(5H)-one with oxalic acid as a coformer for control of animal pests, especially of insects, arachnids and/or nematodes, which occur in agriculture, in forests, in the protection of stored products and of materials, and in the hygiene sector.

In the agricultural sector and in forests, in the context of the use of the inventive cocrystals, it is possible to treat all plants and plant parts.

Plants are understood here to mean all plants and plant populations, such as desired and undesired wild plants or crop plants (including naturally occurring crop plants). Crop plants may be plants which can be obtained by conventional breeding and optimization methods or by biotechnological and genetic engineering methods or combinations of these methods, including the transgenic plants and including the plant varieties which are protectable and non-protectable by plant breeders' rights.

Parts of plants shall be understood to mean all above-ground and below-ground parts and organs of plants, such as shoot, leaf, flower and root, examples including leaves, needles, stems, trunks, flowers, fruit bodies, fruits and seed, and also roots, tubers and rhizomes. The plant parts also include harvested material and vegetative and generative propagation material, for example cuttings, tubers, rhizomes, slips and seed.

The inventive treatment of the plants and plant parts with the inventive cocrystals is effected directly or by allowing them to act on the surroundings, habitat or storage space thereof by the customary treatment methods, for example by dipping, spraying, evaporating, fogging, scattering, painting on, injecting, and, in the case of propagation material, especially in the case of seed, also by applying one or more coats.

Plants which can be treated in the context of the aforementioned use of the inventive cocrystals include the following: cotton, flax, grapevine, fruit, vegetables, such as Rosaceae sp. (for example pome fruits such as apples and pears, but also stone fruits such as apricots, cherries, almonds and peaches, and berry fruits such as strawberries), Ribesioidae sp., Juglandaceae sp., Betulaceae sp., Anacardiaceae sp., Fagaceae sp., Moraceae sp., Oleaceae sp., Actimidaceae sp., Lauraceae sp., Musaceae sp. (for example banana plants and banana plantations), Rubiaceae sp. (for example coffee), Theaceae sp., Sterculiceae sp., Rutaceae sp. (for example lemons, oranges and grapefruit); Solanaceae sp. (for example tomatoes), Liliaceae sp., Asteraceae sp. (for example lettuce), Umbelliferae sp., Cruciferae sp., Chenopodiaceae sp., Cucurbitaceae sp. (for example cucumber), Alliaceae sp. (for example leeks, onions), Papilionaceae sp. (for example peas); major crop plants such as Gramineae sp. (for example maize, turf, cereals such as wheat, rye, rice, barley, oats, millet and triticale), Asteraceae sp. (for example sunflower), Brassicaceae sp. (for example white cabbage, red cabbage, broccoli, cauliflower, Brussels sprouts, pak Choi, kohlrabi, radishes, and also oilseed rape, mustard, horseradish and cress), Fabacae sp. (for example beans, peanuts), Papilionaceae sp. (for example soya beans), Solanaceae sp. (for example potatoes), Chenopodiaceae sp. (for example sugar beet, fodder beet, Swiss chard, beetroot); useful plants and ornamental plants in gardens and forests; and genetically modified types of each of these plants.

The present invention further provides compositions for control of animal pests, especially of insects, arachnids and/or nematodes, which occur in agriculture, in forests, in the protection of stored products and of materials, and in the hygiene sector, which comprise the inventive cocrystals.

Due to their stability, the inventive cocrystals are quite generally suitable as a starting material for the production of any formulated composition comprising the compound 4-{[(6-chloropyrid-3-yl)methyl](2,2-difluoroethyl)amino}furan-2(5H)-one for control of pests, even when the compound 4-{[(6-chloropyrid-3-yl)methyl](2,2-difluoroethyl)amino}furan-2(5H)-one after the formulation is no longer in the form of a cocrystal, but rather, for instance, in dissolved form.

More particularly, the inventive cocrystals are suitable for production of compositions for treatment of seed.

Thus, a large part of the damage to crop plants which is caused by pests arises at an early stage, through infestation of the seed during storage and after the seed has been introduced into the soil, and during and immediately after the germination of the plants. This phase is particularly critical since the roots and shoots of the growing plant are particularly sensitive and even minor damage can lead to the death of the whole plant. There is therefore a particularly great interest in protecting the seed and the germinating plant by using appropriate compositions.

The present invention therefore more particularly also relates to a method for protecting seed, especially transgenic seed, and germinating plants from infestation by pests, in which the seed is treated with one of the inventive compositions.

The invention likewise relates to the use of the inventive compositions for treatment of seed to protect the seed and the plant which arises therefrom from pests. The invention further relates to seed which has been treated with an inventive composition for protection from pests.

As already mentioned above, the treatment of transgenic seed with an inventive composition is also of particular significance. The seed is that of plants which generally comprise at least one heterologous gene which controls the expression of a polypeptide with insecticidal properties in particular. The heterologous genes in transgenic seed can originate from microorganisms such as Bacillus, Rhizobium, Pseudomonas, Serratia, Trichoderma, Clavibacter, Glomus or Gliocladium.

The present invention is particularly suitable for the treatment of transgenic seed which comprises at least one heterologous gene originating from Bacillus sp. and whose gene product shows efficacy against the European corn borer and/or the corn root worm. The gene involved is more preferably a heterologous gene which originates from Bacillus thuringiensis.

One of the advantages of the present invention is that, due to the particular systemic property of the 4-{[(6-chloropyrid-3-yl)methyl](2,2-difluoroethyl)amino}furan-2(5H)-one present in the inventive cocrystals, the treatment of the seed with the inventive compositions protects not just the seed itself, but also the plant originating therefrom after emergence, from pests.

The invention also includes compositions which comprise the inventive cocrystals. Preference is given to compositions which comprise less than 20% by weight of the inventive cocrystals, more preferably less than 15% by weight, even more preferably with less than 10% by weight, especially preferably with less than 5% by weight, most preferably with less than 4, 3, 2 or 1% by weight of the inventive cocrystals in the composition. According to the invention, compositions also comprise the aforementioned compositions.

The inventive cocrystals can be converted in a known manner to the customary formulations, such as suspension concentrates, oil-based suspension concentrates, colloidal concentrates, dispersible concentrates, emulsifiable concentrates (emulsion concentrates), seed-dressing emulsions, seed-dressing suspensions, granules, microgranules, suspoemulsions, water-soluble granules, water-soluble concentrates and water-dispersible granules, using suitable assistants and carriers or solvents.

In this context, the active compound 4-{[(6-chloropyrid-3-yl)methyl](2,2-difluoroethyl)amino}furan-2(5H)-one should be present in a concentration of about 0.5 to 90% by weight of the composition comprising the inventive cocrystals, i.e. in amounts sufficient to achieve the necessary dosage level.

The formulations are produced, for example, by extending the inventive cocrystals with water, solvents and/or carriers, optionally using emulsifiers and/or dispersants, and/or other auxiliaries, for example penetrants.

In the production of suspension concentrates, including those which are used for seed treatment, further assistants are generally added as well as the active ingredient and an extender (water, solvent or oil). A wetting agent is used to moisten the active ingredient in the continuous phase; dispersing aids are used to stabilize the suspension in the liquid phase; emulsifiers are used to emulsify the nonaqueous phase in the case of solvent- or oil-containing suspension concentrates. If necessary, antifreezes, biocides, thickeners, dyes, spreaders and/or uptake promoters are incorporated.

The inventive cocrystals can also be converted to other customary formulations, such as solutions, emulsions, wettable powders, water- and oil-based suspensions, powders, dusts, pastes, soluble powders, soluble granules, granules for broadcasting, suspoemulsion concentrates, cocrystal-impregnated natural products, cocrystal-impregnated synthetic substances, fertilizers and microencapsulations in polymeric substances.

These formulations are produced in a known manner, for example by mixing the inventive cocrystals with extenders, i.e. liquid solvents, and/or solid carriers, optionally with the use of surfactants, i.e. emulsifiers and/or dispersants, and/or foam formers. The formulations are produced either in suitable plants or else before or during application.

The auxiliaries used may be those substances which are suitable for imparting particular properties to the composition itself or and/or to preparations derived therefrom (for example spray liquors, seed dressings), such as certain technical properties and/or also particular biological properties. Typical auxiliaries include: extenders, solvents and carriers.

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulfones and sulfoxides (such as dimethyl sulfoxide).

If the extender utilized is water, it is also possible to use, for example, organic solvents as auxiliary solvents. Useful liquid solvents essentially include: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethyl sulfoxide, and also water.

According to the invention, a carrier is a natural or synthetic, organic or inorganic substance which may be solid or liquid, with which the active ingredients are mixed or combined for better applicability, more particularly for application to plants or plant parts or seed. The solid or liquid carrier is generally inert and should be usable in agriculture.

Useful solid or liquid carriers include, for example, ammonium salts and ground natural minerals such as kaolins, aluminas, talc, chalk, quartz, attapulgite, montmorillonite or diatomaceous earth, and ground synthetic minerals such as finely divided silica, aluminum oxide and silicates. Useful solid carriers for granules include especially, for example, crushed and fractionated natural rocks such as calcite, marble, pumice, sepiolite, dolomite, and synthetic granules of inorganic and organic meals, and also granules of organic material such as paper, sawdust, coconut shells, maize cobs and tobacco stalks.

Useful emulsifiers and/or foam formers include, for example, nonionic and anionic emulsifiers, such as polyoxyethylene fatty acid esters, polyoxyethylene fatty alcohol ethers, for example alkylaryl polyglycol ethers, alkylsulfonates, alkylsulfates, arylsulfonates and protein hydrolyzates.

Useful dispersants include nonionic and/or ionic substances, for example from the classes of the alcohol POE and/or POP ethers, acid and/or POP POE esters, alkylaryl and/or POP POE ethers, fat and/or POP POE adducts, POE- and/or POP-polyol derivatives, POE- and/or POP-sorbitan or -sugar adducts, alkyl- or arylsulfates, alkyl- or arylsulfonates and alkyl- or arylphosphates or the corresponding PO-ether adducts.

Further suitable oligomers or polymers are, for example, those derived from vinylic monomers, from acrylic acid, from EO and/or PO alone or in combination with, for example, (poly)alcohols or (poly)amines. It is also possible to use lignin and sulfonic acid derivatives thereof, unmodified and modified celluloses, aromatic and/or aliphatic sulfonic acids and adducts thereof with formaldehyde.

In the formulations, it is possible to use tackifiers such as carboxymethylcellulose, and natural and synthetic polymers in the form of powders, granules or latices, such as gum arabic, polyvinyl alcohol and polyvinyl acetate, or else natural phospholipids, such as cephalins and lecithins, and synthetic phospholipids.

It is possible to use dyes such as inorganic pigments, for example iron oxide, titanium oxide and Prussian Blue, and organic dyes such as alizarin dyes, azo dyes and metal phthalocyanine dyes, and trace nutrients such as salts of iron, manganese, boron, copper, cobalt, molybdenum and zinc.

Further additives may be perfumes, mineral or vegetable, optionally modified oils, waxes and nutrients (including trace nutrients), such as salts of iron, manganese, boron, copper, cobalt, molybdenum and zinc.

Additional components may be stabilizers, such as cold stabilizers, preservatives, antioxidants, light stabilizers, or other agents which improve chemical and/or physical stability.

The content of 4-{[(6-chloropyrid-3-yl)methyl](2,2-difluoroethyl)amino}furan-2(5H)-one in the use forms prepared from the formulations may vary within wide ranges. The content of 4-{[(6-chloropyrid-3-yl)methyl](2,2-difluoroethyl)amino}furan-2(5H)-one in the use forms is in the range from 0.00000001 to 97% by weight, preferably in the range from 0.0000001 to 97% by weight, more preferably in the range from 0.000001 to 83% by weight or 0.000001 to 5% by weight and most preferably in the range from 0.0001 to 1% by weight.

The inventive cocrystals can optionally, at particular concentrations or application rates, also be used together with herbicides, safeners, growth regulators or agents to improve plant properties, or with microbicides, for example fungicides, especially antimycotics, bactericides, virucides (including compositions against viroids) or with compositions against MLO (Mycoplasma-like organisms) and RLO (Rickettsia-like organisms).

The invention is illustrated in detail hereinafter with reference to examples and figures, but without restricting it thereto.

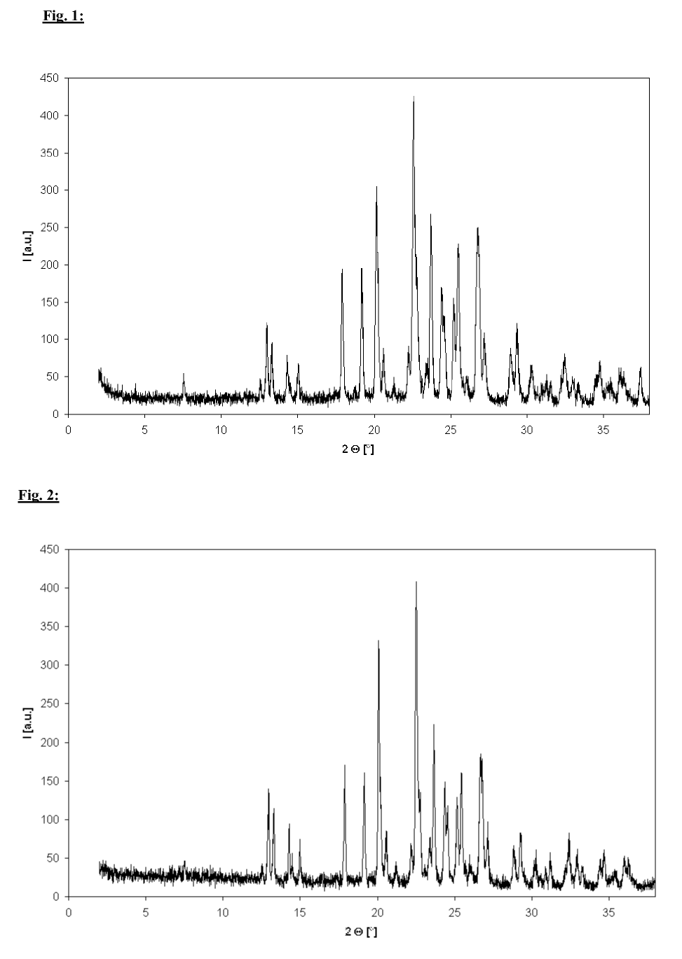

A feature common to FIG. 1 and FIG. 2 which follow is that, in each of these, the intensities of an x-ray [I] determined according to the working examples is shown against twice the diffraction angle measured in each case [2Θ].

FIG. 1 shows an x-ray diffraction analysis of a powder of the inventive cocrystal comprising 4-{[(6-chloropyrid-3-yl)methyl](2,2-difluoroethyl)amino}furan-2(5H)-one and oxalic acid as a coformer according to example 2 in conjunction with example 4.

FIG. 2 shows an x-ray diffraction analysis of a powder of the inventive cocrystal comprising 4-{[(6-chloropyrid-3-yl)methyl](2,2-difluoroethyl)amino}furan-2(5H)-one and oxalic acid as a coformer according to example 3 in conjunction with example 4.

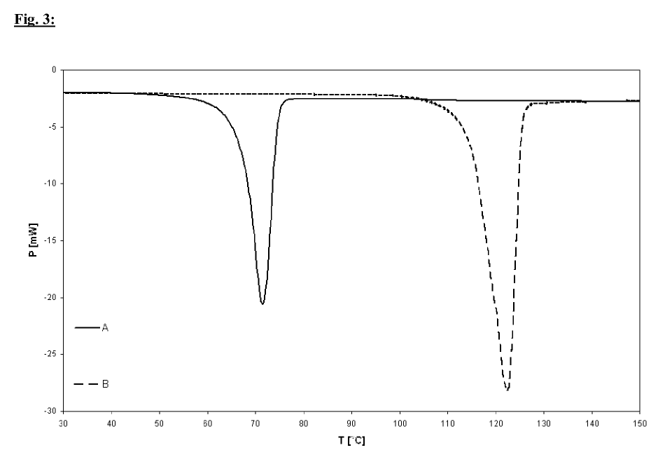

FIG. 3 shows measurement data for differential calorimetry (DSC) according to example 5, plotted as heat capacity (P) absorbed at a temperature (T) for a powder of 4-{[(6-chloropyrid-3-yl)methyl](2,2-difluoroethyl)amino}furan-2(5H)-one according to example 1 (A) and a powder from example 2 (B).

### EXAMPLES

### Example 1

### Preparation of 4-{[(6-chloropyrid-3-yl)methyl](2,2-difluoroethyl)amino}furan-2(5H)-one

4-{[(6-Chloropyrid-3-yl)methyl](2,2-difluoroethyl)amino}furan-2(5H)-one was prepared according to example 2 of WO 2009/036899 A1 and used in examples 2 and 3 to produce the inventive cocrystals.

### Example 2

### Production of a Cocrystal Powder

183 mg of 4-{[(6-chloropyrid-3-yl)methyl](2,2-difluoroethyl)amino}furan-2(5H)-one from example 1 were mixed with 58 mg of oxalic acid in a 2 ml reaction vessel (from Eppendorf). 30 μl of liquid ethyl acetate were added to the mixture. The contents of the aforementioned reaction vessel thus obtained were transferred together with 7 stainless steel balls of diameter 3 mm into a Retsch MM200 vibratory mill and ground therein at 30 Hz for 60 minutes.

The ground material was dried at room temperature (23° C.) and ambient pressure (1013 hPa) overnight, in order to evaporate the ethyl acetate. A powder was obtained, which was supplied to examples 4 and 5.

### Example 3

### Production of a Single Crystal of the Cocrystal

2 ml of a saturated solution of 4-{[(6-chloropyrid-3-yl)methyl](2,2-difluoroethyl)amino}furan-2(5H)-one from example 1 in ethyl acetate were admixed with 2 ml of saturated oxalic acid solution in ethyl acetate and mixed by shaking.

Crystals of size approx. 1 mm formed overnight at room temperature (23° C.) and under ambient pressure (1013 hPa).

Individual single crystals are removed and supplied to the experiment according to example 6. The remaining crystals are manually crushed with a mortar and pestle and supplied to a study according to example 4.

### Example 4

### Powder X-Ray Diffractometry Analysis (p-XRD) of the Powder from Examples 2 and 3

The powder obtained from examples 2 and 3 was analyzed with an x-ray diffractometer (Stoe STADI-P transmission diffractometer, primary monochromator: Ge[1 1 1], radiation source; Cu_{Kα1}, wavelength 1.54 Å, detector: linear PSD). The analysis of the powder from example 2 gave the characteristic x-ray diffractogram shown in FIG. 1. The analysis of the powder from example 3 gave the characteristic x-ray diffractogram shown in FIG. 2.

It is evident that the FIG. 1 and FIG. 2 x-ray diffractograms are of the same substance. Both x-ray diffractograms are different than those of the pure substances 4-{[(6-chloropyrid-3-yl)methyl](2,2-difluoroethyl)amino}furan-2(5H)-one and oxalic acid (neither shown here). Accordingly, both processes according to the invention for production of an inventive cocrystal form the same cocrystal.

### Example 5

### Differential calorimetry (DSC) of the powder from example 2, and of 4-{[(6-chloropyrid-3-yl)methyl](2,2-difluoroethyl)amino}furan-2(5H)-one from example 1

The powder obtained from example 2, and also powder of 4-{[(6-chloropyrid-3-yl)methyl](2,2-difluoroethyl)amino}furan-2(5H)-one, are analyzed in a differential calorimeter (Mettler-Toledo DSC 822). The results of the analyses are shown in FIG. 3.

For each of the powders analyzed, only one melting point is seen in each case, which, in the case of the powder of 4-{[(6-chloropyrid-3-yl)methyl](2,2-difluoroethyl)amino}furan-2(5H)-one, is the melting point of about 72-74° C. of the pure substance, which is altered in the powder from example 2 by the formation of the inventive cocrystal.

The finding that only one melting point was determined in each case shows that the powder from example 2 is not a mixture of the respective pure substances.

The melting point of the cocrystal of 4-{[(6-chloropyrid-3-yl)methyl](2,2-difluoroethyl)amino}furan-2(5H)-one and oxalic acid according to example 2 is about 122° C.

Thus, the formation of the inventive cocrystals increases the melting point of the resulting substance.

### Example 6

### X-Ray Diffraction Analysis (XRD) of the Single Crystal from Example 3

A colorless crystal of approximate dimensions 0.30×0.30×0.06 mm³ from example 3 was analyzed in a diffractometer (Oxford Diffraction Xcalibur) equipped with a CCD area detector (model: Ruby), a Cu_{Kα} radiation source and a Cryojet low-temperature apparatus (T=100 K). The measurement data were recorded in all spatial directions, i.e. the detector was moved completely around the sample in horizontal and vertical angles.

The measurement data were collected and recorded by means of the software Crysalis (Oxford Diffraction 2007).

The solution of the crystal structure equations was executed by means of direct methods, as implemented in the program used, SHELXTL Version 6.10 (Sheldrick, University of Gottingen, Germany, 2000).

This was used to visualize the data, according to which a stoichiometric ratio of 4-{[(6-chloropyrid-3-yl)methyl](2,2-difluoroethyl)amino}furan-2(5H)-one to oxalic acid of 2:1 can be seen.

An overview of the essence of the experiment and measurement data from this example is given in the table which follows.

| Parameter | Value | |
|---|---|---|
| Temperature (measurement) | 120 K | |
| Wavelength (measurement) | 1.54178 Å | |
| Morphology | monoclinic | |
| Space group | C2/c | |
| Dimensions of the unit cell | a = 15.33150(10) Å | α = 90° |
| | b = 7.86280(10) Å | β = 96.2220(10)° |
| | c = 23.1301(2) Å | γ = 90° |
| Volume of the unit cell | 2771.87(5) Å³ | |
| Density (calculated) | 1.599 Mg/m3 | |
| Absorption coefficient | 2.879 mm⁻¹ | |

1. A cocrystal comprising 4-{[(6-chloropyrid-3-yl)methyl](2,2-difluoroethyl)amino}furan-2(5H)-one, wherein said cocrystal comprises oxalic acid as a coformer.

2. The cocrystal as claimed in claim 1, wherein said cocrystal comprises or consists of 4-{[(6-chloropyrid-3-yl)methyl](2,2-difluoroethyl)amino}furan-2(5H)-one and oxalic acid.

3. The cocrystal as claimed in claim 2, wherein the stoichiometric ratio of 4-{[(6-chloropyrid-3-yl)methyl](2,2-difluoroethyl)amino}furan-2(5H)-one to oxalic acid is 2:1.

4. The cocrystal as claimed in claim 2, wherein said cocrystal has monoclinic morphology.

5. The cocrystal as claimed in claim 2, it wherein said cocrystal has the space group C2/c.

6. The cocrystal as claimed in claim 2, wherein said cocrystal has a melting point in a range of 110° C. and 130° C.

7. A process for producing a cocrystal comprising 4-{[(6-chloropyrid-3-yl)methyl](2,2-difluoroethyl)amino}furan-2(5H)-one, wherein said process comprises:
a) admixing 4-{[(6-chloropyrid-3-yl)methyl](2,2-difluoroethyl)amino}furan-2(5H)-one with oxalic acid to obtain a mixture A;
b) optionally further admixing the mixture A with a solvent to obtain a mixture A′; and
c) grinding mixture A or mixture A′ to obtain the cocrystal.

8. A process for producing a cocrystal comprising 4-{[(6-chloropyrid-3-yl)methyl](2,2-difluoroethyl)amino}furan-2(5H)-one, wherein said process comprises:
a) providing a saturated solution (A) of 4-{[(6-chloropyrid-3-yl)methyl](2,2-difluoroethyl)amino}furan-2(5H)-one and/or a saturated solution (B) of oxalic acid[H]; and
b) admixing solutions (A) and (B) and/or or adding at least one oxalic acid as a solid to solution (A) and/or adding 4-{[(6-chloropyrid-3-yl)methyl](2,2-difluoroethyl)amino}furan-2(5H)-one as a solid to solution (B).

9. A cocrystal comprising 4-{[(6-chloropyrid-3-yl)methyl](2,2-difluoroethyl)amino}furan-2(5H)-one with oxalic acid capable of being used as a coformer for control of animal pests.

10. The cocrystal as claimed in claim 9, wherein said animal pests are insects, arachnids and/or nematodes which occur in agriculture, in forests, in protection of stored products and/or of materials, and/or in a hygiene sector.

11. A composition for control of animal pests, which comprises said cocrystalas defined in claim 1.

12. The composition as claimed in claim 11, wherein said animal pests are insects, arachnids and/or nematodes which occur in agriculture, in forests, in protection of stored products and/or of materials, and/or in a hygiene sector.

13. A method for protecting seed and/or germinating plants from infestation by pests, comprising treating the seed with the composition as claimed in claim 11.

14. The method as claimed in claim 13, wherein said seed is a transgenic seed and said pests are insects, arachnids and/or nematodes which occur in agriculture.

15. The cocrystal as claimed in claim 1, wherein said cocrystal consists of 4-{[(6-chloropyrid-3-yl)methyl](2,2-difluoroethyl)amino}furan-2(5H)-one and oxalic acid.

16. The cocrystal as claimed in claim 1, wherein said cocrystal comprises 4-{[(6-chloropyrid-3-yl)methyl](2,2-difluoroethyl)amino}furan-2(5H)-one and oxalic acid.
